(19) United States
(12) Patent Application Publication
(10) Pub. No.: US 2026/0279591 A1
(43) Pub. Date: Sep. 17, 2026

(54) HEALTH CONDITION ESTIMATION SYSTEM, INFORMATION PROCESSING APPARATUS, AND HEALTH CONDITION ESTIMATION METHOD

(71) Applicant: Yokogawa Electric Corporation, Tokyo (JP)

(72) Inventors: Kiyomi Muraoka, Tokyo (JP); Hideo Uemura, Tokyo (JP); Kie Shimamura, Tokyo (JP)

(73) Assignee: Yokogawa Electric Corporation, Tokyo (JP)

(21) Appl. No.: 19/557,354

(22) Filed: Mar. 5, 2026

(30) Foreign Application Priority Data
Mar. 12, 2025 (JP) 2025-039779

(51) Int. Cl. G16H 50/30; G06F 3/01; G16H 20/00; G16H 40/67; G16H 50/20

(52) U.S. Cl. CPC G16H 50/30; G06F 3/015; G16H 20/00; G16H 40/67; G16H 50/20

(57) ABSTRACT

A health condition estimation system (1) according to the present disclosure includes a brain wave sensor (10) configured to detect brain waves of a user and output brain wave information, and an information processing apparatus (20). The information processing apparatus (20) includes a processor (25) that estimates the health condition of the user based on the brain wave information and the attribute information for the user and outputs information on the health condition of the user.

### CROSS-REFERENCE TO RELATED APPLICATION

The present application claims priority to Japanese Patent Application 2025-039779 filed on March 12, 2025, the entire contents of which are incorporated herein by reference.

### TECHNICAL FIELD

The present disclosure relates to a health condition estimation system, an information processing apparatus, a health condition estimation method, and a program.

### BACKGROUND

A technique exists for estimating a health condition based on biological information measured by a sensor.

For example, Patent Literature (PTL) 1 discloses a technology for determining whether a person to be measured is in a resting state and calculating a condition value related to the physical condition of the person to be measured based on sensor information measured when the person to be measured is in a resting state.

### CITATION LIST

### Patent Literature

PTL 1: WO2018/016459

### SUMMARY

A health condition estimation system comprising:

a brain wave sensor configured to detect brain waves of a user and output brain wave information; and

an information processing apparatus, wherein

the information processing apparatus comprises a processor configured to estimate a health condition of the user, based on the brain wave information and attribute information for the user, and output information on the health condition of the user.

An information processing apparatus for estimating a health condition of a user, the information processing apparatus comprising:

a processor configured to

acquire brain wave information from a brain wave sensor that detects brain waves of a user and outputs the brain wave information, and

estimate a health condition of the user, based on the brain wave information and attribute information for the user, and output information on the health condition of the user.

A health condition estimation method comprising:

acquiring brain wave information from a brain wave sensor configured to detect brain waves of a user; and

estimating a health condition of the user, based on the brain wave information and attribute information for the user, and outputting information on the health condition of the user.

A program configured to cause a computer to perform operations comprising:

acquiring brain wave information from a brain wave sensor configured to detect brain waves of a user; and

estimating a health condition of the user, based on the brain wave information and attribute information for the user, and outputting information on the health condition of the user.

### BRIEF DESCRIPTION OF THE DRAWINGS

In the accompanying drawings:

FIG. 1 is a diagram illustrating a schematic configuration of a health condition estimation system according to an embodiment of the present disclosure;

FIG. 2 is a diagram illustrating an example of how a health condition is estimated using an estimation model and an attribute model;

FIG. 3 is a diagram illustrating another example of how a health condition is estimated using an estimation model and an attribute model; and

FIG. 4 is a flowchart illustrating an example of the operation of the health condition estimation system according to an embodiment.

### DETAILED DESCRIPTION

When estimating a health condition based on biological information, demand exists for improving the estimation accuracy.

It would be helpful to provide a health condition estimation system, an information processing apparatus, a health condition estimation method, and a program that are capable of improving estimation accuracy in estimating a health condition based on biological information.

Examples of some embodiments of the present disclosure are described below.

[1] A health condition estimation system comprising:

a brain wave sensor configured to detect brain waves of a user and output brain wave information; and

an information processing apparatus, wherein

the information processing apparatus comprises a processor configured to estimate a health condition of the user, based on the brain wave information and attribute information for the user, and output information on the health condition of the user.

According to such a health condition estimation system, the accuracy of estimation of a health condition based on biological information can be improved.

[2] In the health condition estimation system according to [1],

the information processing apparatus may further comprise a memory configured to store an estimation model created by machine learning based on learning data, and an attribute model created by machine learning based on attribute learning data,

the estimation model may be a model configured to output the information on the health condition when the brain wave information is inputted,

the attribute model may be a model configured to output correction information when the attribute information for the user is inputted, and

the processor may be configured to correct the information on the health condition, outputted by the estimation model, using the correction information and output the corrected information on the health condition.

With this configuration, the information on the health condition outputted by the estimation model created by machine learning can be corrected using the correction information outputted by the attribute model created by machine learning, and the corrected information on the health condition can be outputted.

[3] In the health condition estimation system according to [1] or [2],

the processor may be configured to correct the information on the health condition by adjusting the information on the health condition, outputted by the estimation model, using the correction information.

With this configuration, the information on the health condition can be corrected by adjusting the information on the health condition, outputted by the estimation model, using the correction information.

[4] In the health condition estimation system according to any one of [1] to [3],

the processor may be configured to correct the information on the health condition by adjusting a parameter of the estimation model using the correction information.

With this configuration, the information on the health condition can be corrected by adjusting a parameter of the estimation model using the correction information.

[5] In the health condition estimation system according to any one of [1] to [4],

the information on the health condition may include a stress level or degree of fatigue of the user.

With this configuration, the stress level or degree of fatigue of the user can be estimated.

[6] In the health condition estimation system according to any one of [1] to [5],

the information processing apparatus may further comprise an output interface, and

the processor may be configured to generate an action proposal to propose to the user based on the information on the health condition and display the generated action proposal on the output interface.

With this configuration, appropriate actions can be proposed to the user according to the health condition of the user.

[7] In the health condition estimation system according to any one of [1] to [6],

the processor may be configured to acquire a schedule of the user and generate the action proposal based on the acquired schedule and the information on the health condition.

With this configuration, appropriate actions can be proposed to the user according to the health condition of the user, taking into account the user's schedule.

[8] An information processing apparatus for estimating a health condition of a user, the information processing apparatus comprising:

a processor configured to

acquire brain wave information from a brain wave sensor that detects brain waves of a user and outputs the brain wave information, and

estimate a health condition of the user, based on the brain wave information and attribute information for the user, and output information on the health condition of the user.

According to such an information processing apparatus, the accuracy of estimation of a health condition based on biological information can be improved.

[9] A health condition estimation method comprising:

acquiring brain wave information from a brain wave sensor configured to detect brain waves of a user; and

estimating a health condition of the user, based on the brain wave information and attribute information for the user, and outputting information on the health condition of the user.

According to such a health condition estimation method, the accuracy of estimation of a health condition based on biological information can be improved.

A program configured to cause a computer to perform operations comprising:

acquiring brain wave information from a brain wave sensor configured to detect brain waves of a user; and

estimating a health condition of the user, based on the brain wave information and attribute information for the user, and outputting information on the health condition of the user.

According to such a program, the accuracy of estimation of a health condition based on biological information can be improved.

According to the present disclosure, a health condition estimation system, an information processing apparatus, a health condition estimation method, and a program that are capable of improving estimation accuracy in estimating a health condition based on biological information can be provided.

An embodiment of the present disclosure will be described below, with reference to the drawings.

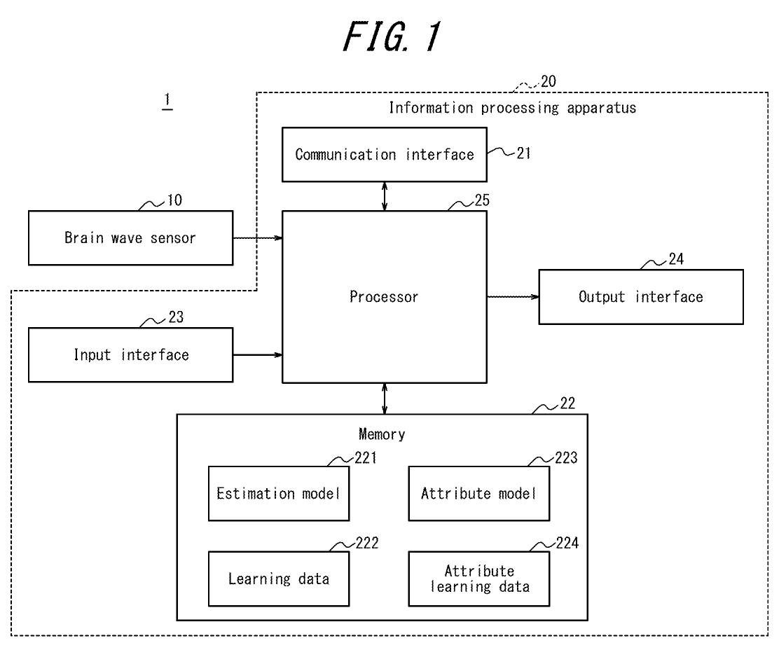

FIG. 1 is a diagram illustrating a schematic configuration of a health condition estimation system 1 according to an embodiment of the present disclosure. The health condition estimation system 1 includes a brain wave sensor 10 and an information processing apparatus 20. The brain wave sensor 10 and the information processing apparatus 20 can communicate by wireless communication or wired communication.

The health condition estimation system 1 can estimate the health condition of a user who operates the information processing apparatus 20.

The brain wave sensor 10 is a sensor that detects brain waves of a user operating the information processing apparatus 20 and outputs brain wave information. Upon detecting brain waves of the user, the brain wave sensor 10 transmits brain wave information to the information processing apparatus 20.

The brain wave sensor 10 is attached to a user who is operating the information processing apparatus 20, and is thereby capable of detecting the brain waves of the user. The brain wave sensor 10 may be any brain wave sensor capable of detecting the brain waves of the user. The brain wave sensor 10 may be an earphone-type brain wave sensor, but this example is not limiting.

The information processing apparatus 20 is a device used by a user whose health condition is to be estimated. The information processing apparatus 20 can estimate the health condition of the user by using the brain wave information acquired from the brain wave sensor 10.

The information processing apparatus 20 may be a general-purpose electronic device such as a PC (Personal Computer) or a tablet terminal.

The information processing apparatus 20 includes a communication interface 21, a memory 22, an input interface 23, an output interface 24, and a processor 25.

The communication interface 21 includes a communication module. For example, the communication interface 21 may include a communication module compatible with a LAN (Local Area Network). In one embodiment, the information processing apparatus 20 is connected to a network via the communication interface 21. The communication interface 21 may be capable of communicating with a server or the like via the network.

The memory 22 is, for example, a semiconductor memory, a magnetic memory, an optical memory, or the like, but is not limited to these. The memory 22 may function as, for example, a main memory device, an auxiliary memory device, or a cache memory. The memory 22 stores any information used in the operation of the information processing apparatus 20. For example, the memory 22 may store system programs, application programs, various types of information, and the like. A portion of the memory 22 may be installed outside the information processing apparatus 20. In this case, a portion of the externally installed memory 22 may be connected to the information processing apparatus 20 via any appropriate interface.

The input interface 23 includes one or more interfaces for input that receive an operation from the user and acquire input information based on the user operation. For example, the input interface 23 includes at least one of a keyboard, a mouse, and a touch screen that is integrated with the display of the output interface 24, but is not limited to these.

The output interface 24 includes one or more interfaces for output that output information to notify the user. For example, the output interface 24 includes a display that outputs information as an image, a speaker that outputs information as sound, and the like, but is not limited to these.

The processor 25 is a general-purpose processor, such as a Central Processing Unit (CPU) or a Graphics Processing Unit (GPU), or a dedicated processor that is dedicated to specific processing. The processor 25 may be a Field-Programmable Gate Array (FPGA), a Digital Signal Processor (DSP), an Application Specific Integrated Circuit (ASIC), or the like. The processor 25 may be a combination of a plurality of processors. The processor 25 executes processes related to the operation of the information processing apparatus 20 while controlling each component of the information processing apparatus 20.

The functions of the information processing apparatus 20 can be realized by causing the processor 25 to execute a program stored in the memory 22. That is, the functions of the information processing apparatus 20 can be realized by software.

### Operation of health condition estimation system

Next, the operation of the health condition estimation system 1 will be described.

The brain wave sensor 10 detects the brain waves of a user who is operating the information processing apparatus 20. Upon detecting brain waves of the user, the brain wave sensor 10 transmits brain wave information to the information processing apparatus 20.

The processor 25 of the information processing apparatus 20 acquires the brain wave information transmitted by the brain wave sensor 10. When the brain wave sensor 10 and the communication interface 21 are communicating with each other, the processor 25 may acquire the brain wave information transmitted by the brain wave sensor 10 via the communication interface 21.

The processor 25 acquires attribute information for the user operating the information processing apparatus 20. The processor 25 may, for example, acquire the attribute information for the user via an input operation by the user on the input interface 23. The processor 25 may acquire the attribute information for the user by presenting the user with questions regarding attributes of the user and accepting the answers from the user via the input interface 23 during user registration. When the attributes of the user change, the processor 25 may acquire the change in the attribute information for the user by the user self-reporting. The processor 25 may periodically request that the user input the attribute information. The processor 25 may acquire the attribute information for the user from another device via the communication interface 21. Hereinafter, attribute information for the user may be simply referred to as "attribute information".

The attribute information is information that represents the characteristics of a user. The attributes are used, for example, to classify users having the same attributes into a group. In the present embodiment, the attribute information may include information that can reveal some tendency or commonality in the correlation between brain wave information and information on the health condition for users with the same attribute.

The attribute information may include, for example, the age, sex, physical characteristics, or personality tendencies of the user. Age may be expressed as the numerical value of age itself, or may be expressed by classification into age groups such as 40s or categories such as young, middle-aged, or elderly. The physical characteristics include the height, weight, obesity level, and the like of the user and may be expressed by classifying the numerical value of the height, weight, obesity level, and the like into a plurality of categories. The physical characteristics may be expressed as a combination of numerical values such as height, weight, or obesity level. The personality tendencies may be expressed by classification into tendencies such as calm, emotional, introverted, and extroverted.

The attribute information may include, for example, the work style of the user. The work style may be classified by whether the employee works in the office, at home, or the like. The work style may be classified as fixed working hours, flextime, reduced working hours, or the like.

The attribute information may include, for example, the occupation of the user. The occupation may be classified as desk work, manual labor, or the like.

The attribute information may include, for example, the job position of the user. The job position may be expressed by classification into superior or subordinate, or may be expressed by classification into a hierarchy such as a section chief or department chief.

The attribute information is not limited to the examples described above and may include various other characteristics.

As illustrated in FIG. 1, the memory 22 stores an estimation model 221, learning data 222, an attribute model 223, and attribute learning data 224. In the example illustrated in FIG. 1, the memory 22 stores the learning data 222 and the attribute learning data 224, but this is just an example. The learning data 222 and the attribute learning data 224 may be stored in a device other than the information processing apparatus 20, for example.

The estimation model 221 is a model for estimating the health condition of the user based on the brain wave information acquired from the brain wave sensor 10. When the brain wave information is inputted, the estimation model 221 estimates the health condition of the user and outputs information on the health condition of the user.

The estimation model 221 may be a model trained by machine learning based on the learning data 222. The learning data 222 is a data set used by the estimation model 221 when performing machine learning. The learning data 222 may be a data set that associates past brain wave information on the user with past information on the health condition of the user. In a case in which the estimation model 221 is a machine-learned model, the accuracy of estimating the health condition improves as the amount of accumulated past data increases.

The estimation model 221 is not limited to a machine-learned model. The estimation model 221 may, for example, be a regression model.

The attribute model 223 is a model that outputs correction information when attribute information is inputted. The correction information is information used to correct the information on the health condition outputted by the estimation model 221.

Even if the brain wave information is the same, the health condition of the user may differ depending on the attributes of the user. For example, even if the brain wave information is the same, the healthy state of the user may differ depending on whether the personality of the user tends to be calm or emotional. By correcting the information on the health condition outputted by the estimation model 221 with the correction information outputted by the attribute model 223, the processor 25 can estimate the health condition of the user taking into account the attributes of the user, thereby improving the estimation accuracy when estimating the healthy state of the user.

The attribute model 223 may be a machine-learned model based on the attribute learning data 224. The attribute learning data 224 is a data set used by the attribute model 223 when performing machine learning. The attribute learning data 224 may be a data set that associates the attributes of the user with past brain wave information and information on the health condition for users who belong to the group classified by those attributes. In a case in which the attribute model 223 is a machine-learned model, the accuracy of the correction information improves as the amount of accumulated past data increases.

The attribute model 223 is not limited to a machine-learned model. The attribute model 223 may, for example, be a regression model.

Upon acquiring the brain wave information from the brain wave sensor 10, the processor 25 estimates the health condition of the user in real time based on the brain wave information and the attribute information and outputs information on the health condition of the user. The processor 25 may acquire the attribute information for the user at the same time as acquiring the brain wave information or may acquire the attribute information for the user in advance.

The process in which the processor 25 outputs information on the health condition of the user based on the brain wave information and attribute information will be described in more detail.

The processor 25 uses the correction information outputted by the attribute model 223 to correct the information on the health condition outputted by the estimation model 221 and outputs the corrected information on the health condition.

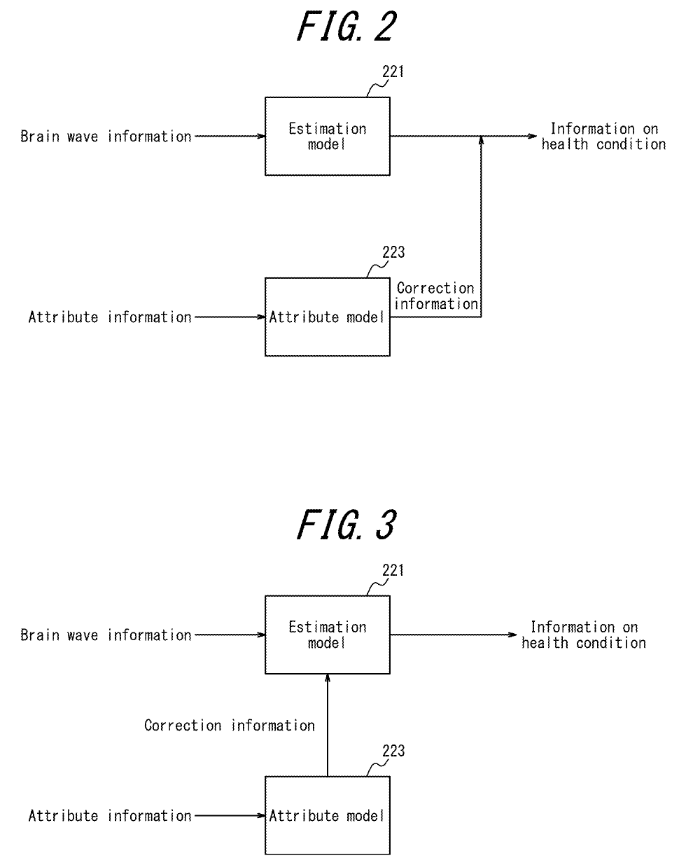

FIG. 2 illustrates an example of how the information on the health condition outputted by the estimation model 221 is corrected using the correction information outputted by the attribute model 223.

In the example illustrated in FIG. 2, when the processor 25 inputs attribute information to the attribute model 223, the attribute model 223 outputs correction information. The processor 25 corrects the information on the health condition by using the correction information outputted by the attribute model 223 to adjust the information on the health condition outputted by the estimation model 221. The processor 25 outputs the information on the health condition corrected in this way.

FIG. 3 illustrates another example of how the information on the health condition outputted by the estimation model 221 is corrected using the correction information outputted by the attribute model 223.

In the example illustrated in FIG. 3, when the processor 25 inputs attribute information to the attribute model 223, the attribute model 223 outputs correction information. The processor 25 corrects the information on the health condition by using the correction information outputted by the attribute model 223 to adjust a parameter of the estimation model 221. The processor 25 outputs the information on the health condition corrected in this way. The parameter of the estimation model 221 to be adjusted by the correction information may be any parameter in the estimation model 221. For example, the parameter may be a weighting parameter.

The processor 25 may correct the information on the health condition outputted by the estimation model 221 by either the method illustrated in FIG. 2 or the method illustrated in FIG. 3.

The information on the health condition of the user outputted by the processor 25 includes various information relating to the health condition. The information on the health condition may, for example, include the stress level or degree of fatigue of the user. The stress level of the user may, for example, be a numerical representation of the stress on the user. The degree of fatigue of the user may, for example, be a numerical representation of the degree to which the user is fatigued.

Upon estimating the health condition of the user, the processor 25 may cause the output interface 24 to display information on the estimated health condition of the user. This enables users to grasp information on their own health condition in real time.

The processor 25 may also generate an action proposal to propose to the user based on the information on the estimated health condition. For example, if the information on the health condition indicates that the user has a high stress level or degree of fatigue, an action proposal such as taking a break, stretching, exercising, or finishing work early may be generated.

Upon generating the action proposal, the processor 25 may cause the output interface 24 to display the action proposal. This enables users to take appropriate actions according to their own health condition and reduce their stress level or degree of fatigue.

When displaying the action proposal on the output interface 24, the processor 25 may display the action proposal together with the information on the health condition. This allows users to check their own health condition and appropriate actions at the same time.

When generating the action proposal, the processor 25 may acquire the schedule of the user and generate the action proposal based on the acquired schedule and the information on the estimated health condition. In this case, the processor 25 may acquire the schedule of the user from a terminal device such as a smartphone used by the user, or may acquire the schedule of the user from a server that stores the schedule of the user.

For example, when the schedule of the user includes a meeting, the processor 25 may generate an action proposal to reduce the stress level or degree of fatigue before the start of the meeting, such as, "You have a meeting at 4 p.m. Why not take a break and refresh yourself before the meeting?"

This enables the processor 25 to generate appropriate action proposals according to the schedule of the user.

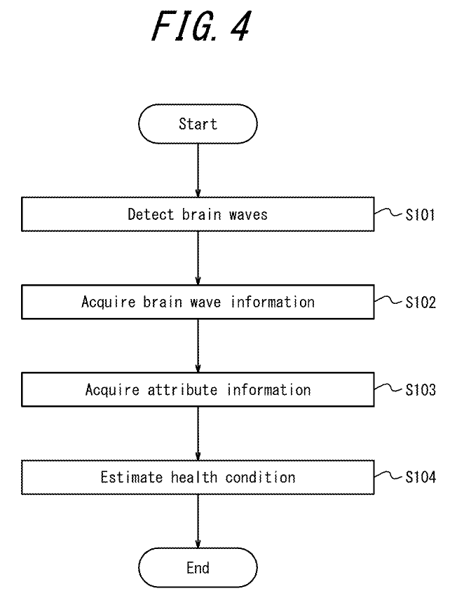

The operation of the health condition estimation system 1 will be described with reference to the flowchart illustrated in FIG. 4.

Step S101: The brain wave sensor 10 detects brain waves of the user who is operating the information processing apparatus 20. Upon detecting brain waves of the user, the brain wave sensor 10 transmits brain wave information to the information processing apparatus 20.

Step S102: The processor 25 of the information processing apparatus 20 acquires the brain wave information transmitted by the brain wave sensor 10.

Step S103: The processor 25 acquires attribute information based on an input operation by the user to the input interface 23 of the information processing apparatus 20. Note that step S103 may be executed before step S101.

Step S104: Upon acquiring the brain wave information and the attribute information, the processor 25 estimates the health condition of the user based on the brain wave information and the attribute information. The processor 25 may cause the output interface 24 to display the information on the estimated health condition of the user.

According to the health condition estimation system 1 of the embodiment as described above, the estimation accuracy in estimating a health condition based on biological information can be improved. More specifically, the health condition estimation system 1 includes a brain wave sensor 10 that detects brain waves of the user and outputs brain wave information, and an information processing apparatus 20. The information processing apparatus 20 includes a processor 25 that estimates the health condition of the user based on the brain wave information and the attribute information for the user and outputs information on the health condition of the user. In this way, the information processing apparatus 20 estimates the health condition of the user by taking into consideration not only the brain wave information, which is the biological information for the user, but also attribute information about the attributes of the user. Therefore, according to the health condition estimation system 1 of the present embodiment, it is possible to improve the estimation accuracy in estimating a health condition based on biological information.

Furthermore, the health condition estimation system 1 according to the present embodiment may generate an action proposal to propose to the user based on the information on the health condition and cause the generated action proposal to be displayed on the output interface 24. If the user responds by implementing the suggested action, the stress on the user can be reduced, and the concentration and productivity of the user can be improved.

It will be apparent to those skilled in the art that the present disclosure may be realized in certain forms other than the above embodiments without departing from the spirit or essential characteristics of the present disclosure. Accordingly, the foregoing description is merely illustrative and is not limiting. The scope of the disclosure is defined by the appended claims, not by the foregoing description. Among all modifications, those within a range of equivalents to the present disclosure shall be considered as being included in the present disclosure.

For example, the arrangement and number of each of the above-mentioned components are not limited to those illustrated in the above description and the drawings. The arrangement and number of each component may be configured freely as long as the corresponding function can be realized.

For example, a general-purpose electronic device such as a smartphone or a computer may be configured to function as the information processing apparatus 20 according to the above embodiment. Specifically, a program describing the processing details to realize each function of the information processing apparatus 20 according to the embodiment could be stored in the memory of the electronic device, and the program could be read and executed by the processor of the electronic device. An embodiment of the present disclosure can thus also be realized as a program executable by a processor.

For example, part of the processing executed by the information processing apparatus 20 in the above embodiment may be executed on a server capable of communicating with the information processing apparatus 20. For example, the information processing apparatus 20 may transmit the brain wave information and the attribute information to the server, and the server may execute processing to estimate the health condition of the user based on the brain wave information and attribute information acquired from the information processing apparatus 20.

For example, in the above embodiment, the information processing apparatus 20 acquires brain wave information, but the information processing apparatus 20 may acquire biological information other than brain wave information together with the brain wave information. The biological information other than brain waves may, for example, be heart rate, blood pressure, or body temperature.

For example, in the above embodiment, the information processing apparatus 20 estimates the health condition of a user who is operating the information processing apparatus 20, but the information processing apparatus 20 may estimate the health condition of a user who is not operating the information processing apparatus 20.

1. A health condition estimation system comprising:
a brain wave sensor configured to detect brain waves of a user and output brain wave information; and
an information processing apparatus, wherein
the information processing apparatus comprises a processor configured to estimate a health condition of the user, based on the brain wave information and attribute information for the user, and output information on the health condition of the user.

2. The health condition estimation system according to claim 1, wherein
the information processing apparatus further comprises a memory configured to store an estimation model created by machine learning based on learning data, and an attribute model created by machine learning based on attribute learning data,
the estimation model is a model configured to output the information on the health condition when the brain wave information is inputted,
the attribute model is a model configured to output correction information when the attribute information for the user is inputted, and
the processor is configured to correct the information on the health condition, outputted by the estimation model, using the correction information and output the corrected information on the health condition.

3. The health condition estimation system according to claim 2, wherein the processor is configured to correct the information on the health condition by adjusting the information on the health condition, outputted by the estimation model, using the correction information.

4. The health condition estimation system according to claim 2, wherein the processor is configured to correct the information on the health condition by adjusting a parameter of the estimation model using the correction information.

5. The health condition estimation system according to claim 1, wherein the information on the health condition includes a stress level or degree of fatigue of the user.

6. The health condition estimation system according to claim 1, wherein
the information processing apparatus further comprises an output interface, and
the processor is configured to generate an action proposal to propose to the user based on the information on the health condition and displays the generated action proposal on the output interface.

7. The health condition estimation system according to claim 6, wherein the processor is configured to acquire a schedule of the user and generate the action proposal based on the acquired schedule and the information on the health condition.

8. An information processing apparatus for estimating a health condition of a user, the information processing apparatus comprising:
a processor configured to
acquire brain wave information from a brain wave sensor that detects brain waves of a user and outputs the brain wave information, and
estimate a health condition of the user, based on the brain wave information and attribute information for the user, and output information on the health condition of the user.

9. A health condition estimation method comprising:
acquiring brain wave information from a brain wave sensor configured to detect brain waves of a user; and
estimating a health condition of the user, based on the brain wave information and attribute information for the user, and outputting information on the health condition of the user.
